(19) United States
(12) Patent Application Publication
(10) Pub. No.: US 2026/0123966 A1
(43) Pub. Date: May 7, 2026

(54) ADJUSTABLE IMPLANT

(71) Applicant: NuVasive Specialized Orthopedics, INC., San Diego, CA (US)

(72) Inventors: Jorge Lopez Camacho, Oxnard, CA (US); Shawn Placie, Aliso Viejo, CA (US)

(21) Appl. No.: 19/437,579

(22) Filed: Dec. 31, 2025

Related U.S. Application Data
(63) Continuation of application No. 18/142,762, filed on May 3, 2023, now Pat. No. 12,533,164.

(51) Int. Cl. A61B 17/72; A61B 17/00; A61B 17/66; A61B 17/68; A61B 90/00

(52) U.S. Cl. CPC A61B 17/72; A61B 17/66; A61B 17/7216; A61B 2017/00398; A61B 2017/00734; A61B 2017/681; A61B 2090/064

(57) ABSTRACT

One aspect of the disclosure relates to an aspect of the disclosure relates to an adjustable implant including: a housing; a first adjustable member at least partially positioned within the housing and moveable relative to the housing; and a first actuation assembly positioned within the first adjustable member and configured to move the first adjustable member relative to the housing.

### CROSS-REFERENCE TO RELATED APPLICATION(S)

This patent application is a continuation of U.S. patent application Ser. No. 18/142,762, filed May 3, 2023, which is incorporated by reference in their entireties for all purposes.

### TECHNICAL FIELD

The subject matter described herein relates to adjustable implants, including intramedullary distraction and compression devices and/or adjustable spinal rods.

### BACKGROUND

Distraction osteogenesis is a technique which has been used to grow new bone in patients with a variety of defects. For example, limb lengthening is a technique in which the length of a bone, for example a femur or tibia, may be increased. By creating a corticotomy or osteotomy in the bone, which is a cut through the bone, the two resulting sections of bone may be moved apart at a particular rate, such as one (1.0) mm per day, allowing new bone to regenerate between the two sections as they move apart. This technique of limb lengthening may be used in cases where one limb is longer than the other, such as in a patient whose prior bone break did not heal correctly, or in a patient whose growth plate was diseased or damaged prior to maturity. In some patients, stature lengthening is desired, and is achieved by lengthening both femurs and/or both tibias to increase the patient's height.

Limb lengthening is often performed using external fixation, wherein an external distraction frame is attached to the two sections of bone by pins which pass through the skin. The pins can be sites for infection and are often painful for the patient, as the pin placement site or “pin tract” remains a somewhat open wound throughout the treatment process. The external fixation frames are also bulky, making it difficult for patient to comfortably sit, sleep, and move. Intramedullary adjustable implants such as lengthening devices also exist, such as those described in U.S. Patent Application Publication No. 2011/0060336, which is incorporated by reference herein.

However, certain conventional intramedullary adjustable implants can be bulky, making them impractical for smaller bones. Further, it can be difficult to precisely determine the load on certain conventional intramedullary adjustable implants, e.g., through indirect load measurement on pins. Even further, certain conventional intramedullary adjustable implants can suffer from communication-based issues such as signal interference from the implant components.

### SUMMARY

All examples and features mentioned below can be combined in any technically possible way.

A first aspect of the disclosure provides an adjustable implant including: a housing having an internal thread and defining a cavity within the housing; an adjustable member at least partially positioned within the housing and moveable relative to the housing within the cavity; an actuation assembly positioned within the adjustable member and configured to move the adjustable member relative to the housing, the actuation assembly including: an actuator configured to be activated by an external adjustment device; a gear assembly coupled to the actuator; and an output driver coupled to the gear assembly; and a sleeve positioned radially between the actuation assembly and the adjustable member, the sleeve configured to move independently of the adjustable member to enable measurement of a load on the adjustable implant.

A second aspect of the disclosure provides an adjustable implant including: a housing having an internal thread and defining a cavity within the housing; an adjustable member at least partially positioned within the housing and moveable relative to the housing within the cavity, the adjustable member including a transducer adjacent an outer wall thereof; an actuation assembly positioned within the adjustable member and configured to move the adjustable member relative to the housing, the actuation assembly including: an actuator configured to be activated by an external adjustment device; a gear assembly coupled to the actuator; and an output driver coupled to the gear assembly; and a sleeve located radially between the actuation assembly and the adjustable member, the sleeve including a load cell coupled with the transducer.

In certain implementations, the sleeve surrounds the actuation assembly.

In certain implementations, the sleeve includes a load cell contained in the adjustable member.

In certain implementations, the load cell is coupled with an end of the actuation assembly.

In certain implementations, the load cell permits measurement of the load on the adjustable implant from inside the adjustable implant.

In certain implementations, the load cell permits measurement of the load on the adjustable implant without a direct measurement of load on a bone anchor coupling the adjustable implant with a bone of a patient.

In certain implementations, the load cell permits measurement of both a compressive load and a tensile load on the adjustable implant.

In certain implementations, the implant further includes a strain gage connected with the load cell by a set of interconnects.

In certain implementations, the strain gage includes a circuit for calculating a load value from detected strain.

In certain implementations, the strain gauge is coupled with a transducer for transmitting load value data from the adjustable implant.

In certain implementations, the implant further includes an alignment pin coupling the strain gauge with the load cell.

In certain implementations, the adjustable member includes a shoulder engaged with the alignment pin.

In certain implementations, the implant further includes a retainer coupling the load cell with the adjustable member.

In certain implementations, the implant further includes an anti-rotation lug at least partially surrounding the adjustable member for limiting rotation of the adjustable member with respect to the housing.

In certain implementations, the housing includes a set of threads having distinct thread pitches to control a distraction rate of the actuation assembly.

In certain implementations, the actuator includes a magnetic actuator.

In certain implementations, the implant further includes a locking mechanism coupled with the gear assembly for controlling distraction loss of the actuation assembly.

In certain implementations, the actuator includes a motor with an integrated distraction loss mechanism.

In certain implementations, the sleeve includes an anti-rotation mechanism that enables the sleeve to move axially independently of the adjustable member without substantial rotation about a distraction axis.

In certain implementations, the implant further includes an anti-jam member in the cavity.

In certain implementations, the gear assembly includes a ring gear comprising an anti-rotation feature.

In certain implementations, the gear assembly includes a four-stage gear assembly.

In certain implementations, the implant further includes a battery slot in the adjustable member and a battery in the battery slot.

In certain implementations, the implant further includes a transducer connected with the battery in the adjustable member.

In certain implementations, the transducer is positioned such that the housing does not obstruct signal transmission to or from the transducer while the adjustable implant is in a fully retracted state.

In certain implementations, the transducer includes an ultrasound transducer.

In certain implementations, the ultrasound transducer includes a piezoelectric transducer.

In certain implementations, the piezoelectric transducer is partially arcuate to enable directional control of ultrasonic signals.

In certain implementations, the partially arcuate piezoelectric transducer has a tuned signal strength in at least one direction.

In certain implementations, the piezoelectric transducer abuts an outer wall of the adjustable member.

In certain implementations, the piezoelectric transducer is bonded directly to an inner surface of the outer wall of the adjustable member without an air gap therebetween, or the piezoelectric transducer is bonded to the inner surface of the outer wall of the adjustable member with a titanium sleeve therebetween.

In certain implementations, a method includes: positioning the piezoelectric transducer in the adjustable member.

In certain implementations, the method further includes bonding the piezoelectric transducer to an inner surface of an outer wall of the adjustable member.

In certain implementations, a method includes measuring the load on the adjustable implant.

In certain implementations, the method further includes actuating the actuator to adjust the adjustable implant based on the measured load.

In certain implementations, the transducer is configured to transmit signals about a load on the adjustable implant detected by the load cell.

In certain implementations, the sleeve is configured to move independently of the adjustable member to enable measurement of a load on the adjustable implant.

In certain implementations, the implant further includes a battery slot in the adjustable member and a battery in the battery slot.

In certain implementations, the transducer is connected with the battery.

In certain implementations, the transducer is positioned such that the housing does not obstruct signal transmission to or from the transducer while the adjustable implant is in a fully retracted state.

In certain implementations, a method includes: positioning the piezoelectric transducer in the adjustable member.

In certain implementations, the method further includes bonding the piezoelectric transducer to an inner surface of an outer wall of the adjustable member.

In certain implementations, the method further includes measuring the load on the adjustable implant.

In certain implementations, the method further includes actuating the actuator to adjust the adjustable implant based on the measured load.

Two or more features described in this disclosure, including those described in this summary section, may be combined to form implementations not specifically described herein.

The details of one or more implementations are set forth in the accompanying drawings and the description below. Other features, objects and benefits will be apparent from the description and drawings, and from the claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a cross-sectional view of an adjustable implant in an extended position according to embodiments of the disclosure;

FIG. 2 shows the adjustable implant of FIG. 1 in a retracted position;

FIG. 3 is a partial sectional view of a portion of an adjustable implant according to embodiments of the disclosure;

FIG. 4 is a cross-sectional view of an adjustable implant according to embodiments of the disclosure;

FIG. 5 is a perspective cut-away view of an adjustable implant according to embodiments of the disclosure;

FIG. 6 illustrates a transducer in an adjustable implant in a first position, according to embodiments of the disclosure;

FIG. 7 illustrates the transducer in the implant of FIG. 6 in a second position;

FIG. 8 is an end cross-sectional view of an adjustable implant illustrating bonding of a transducer to a wall according to embodiments of the disclosure;

FIGS. 9-11 illustrate features of a distraction loss mechanism in an adjustable implant according to embodiments of the disclosure;

FIGS. 12-14 illustrate anti-rotation and anti-jam features in an adjustable implant according to embodiments of the disclosure;

FIG. 15 shows internal components of an external adjustment device for non-invasively adjusting an adjustable implant according to embodiments of the disclosure;

FIGS. 16 and 17 show external adjustment devices in configurations for adjusting an adjustable implant that is implanted within a femur, and within a tibia, respectively, in accordance with embodiments of the disclosure.

It is noted that the drawings of the subject matter are not necessarily to scale. The drawings are intended to depict only typical aspects of the subject matter, and therefore, should not be considered as limiting the scope of the disclosed subject matter. In the drawings, like numbering represents like elements between the drawings.

### DETAILED DESCRIPTION

The present disclosure describes various embodiments of an adjustable implant, such embodiments including intramedullary distraction and compression devices and/or adjustable spinal rods. The adjustable implant is configured to be externally controlled by an external adjustment device, and is therefore non-invasively adjustable. The adjustable implant has a compact size to enable adjustment (e.g., lengthening) of small bones. Further, the adjustable implant can be configured to enable precise load measurement while mitigating interference from implant components. In particular implementations, the adjustable implant can include a sleeve positioned between an actuation assembly and an adjustable member, where the sleeve is configured to move independently of the adjustable member to enable measurement of a load on the adjustable implant. In certain cases, the sleeve includes a load cell that permits measurement of the load on the adjustable implant from inside the implant, e.g., without direct measurement on a bone anchor. The adjustable implant can further include an ultrasound transducer for communicating load data with an external device.

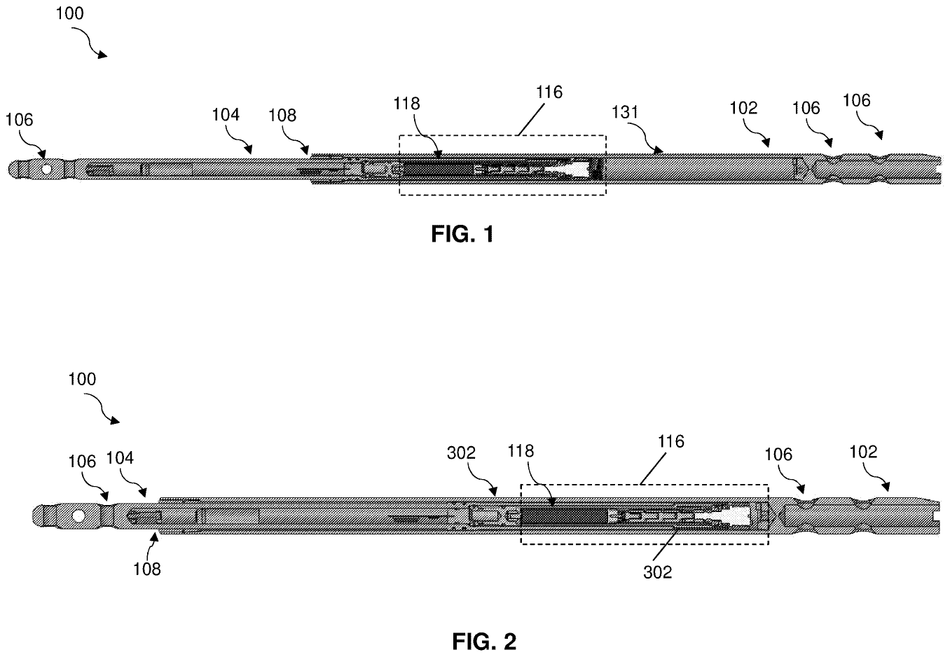

FIGS. 1 and 2 show an adjustable implant 100 according to various implementations. FIG. 1 shows the adjustable implant 100 in an approximately fully extended position, and FIG. 2 shows the adjustable implant 100 in an approximately fully retracted position. In these embodiments, the adjustable implant 100 includes a housing 102 and an adjustable member 104. The adjustable member 104 is at least partially positioned within the housing 102 and is moveable relative to the housing 102. As shown, each of the housing 102 and adjustable member 104 can include one or more fixation apertures 106 for receiving fixation elements, e.g., bone screws, therein for affixing the housing 102 and the adjustable member 104 to respective sections of bone.

The housing 102 is configured to be affixed to a bone at a first location, and the adjustable member 104 is configured to be affixed to the bone at a second location. The first and second locations may be disposed on separate sections of the same bone, or on separate bones, e.g., in the case of a spinal rod. In order to grow or lengthen the bone, the bone either has a pre-existing separation or is purposely cut or broken (e.g., via an osteotomy) to create this separation, dividing the bone into a first section and a second section. The cut may be done prior to implanting and securing the adjustable implant 100 or may be done after the adjustable implant 100 is implanted, for example by use of a flexible Gigli saw. As described herein, the implant 100 is configured such that the adjustable member 104 can contract (e.g., for compression) and/or distract (e.g., for limb lengthening) relative to the housing 102. The adjustable implant 100 is configured to provide controlled, precise translation of the adjustable member 104 relative to the housing 102 by non-invasive remote control, and thus provide controlled, precise translation of the bone segment that is secured to the adjustable member 104 relative to the bone segment that is secured to the housing 102.

Over the treatment period for limb lengthening, the bone is regularly distracted, creating a new separation, into which osteogenesis can occur. “Regularly distracted” is meant to indicate that distraction occurs on a regular or periodic basis which may be on the order of every day or every few days. An exemplary distraction rate is one millimeter per day, although, other distraction rates may be employed. That is to say, a typical distraction regimen may include a daily increase in the length of the adjustable implant 100 by about one millimeter. This may be done, for example, by four lengthening periods per day, each having 0.25 mm of lengthening. The adjustable implant 100, as disclosed in more detail below, may include a magnetic drive system, which causes the adjustable member 104 to telescopically extend from the housing 102, thus forcing the first section and the second section of the bone apart from one another.

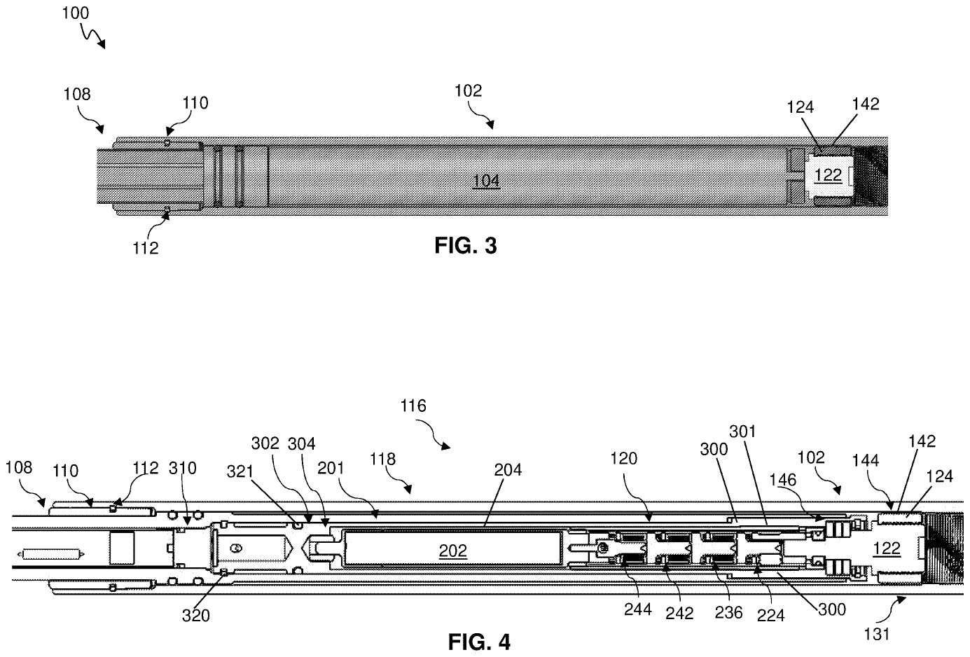

Turning to FIG. 3, at one end, the housing 102 has an opening 108 for receiving the adjustable member 104 in a cavity 131 of the housing 102. One or more o-rings, radial seals, or retainers can be positioned about the adjustable member 104 between the adjustable member 104 and the housing 102. In some embodiments, an intermediary member 110 is positioned between the housing 102 and the adjustable member 104. The intermediary member 110 provides for improved sealing and/or retention. Specifically, in some cases, the intermediary member 110 can provide an anti-rotation mechanism that prevents rotation of the adjustable member 104 relative to the housing 102. For example, an outer geometry of the adjustable member 104 and an inner geometry of the intermediary member 110 may complement and matingly engage one another. For example, the adjustable member 104 may have a cross sectional geometry that is oblong, elliptical, etc., and the opening 108 of the housing 102 may have a cross sectional geometry that is complementary to that of the adjustable member 104 and is therefore also oblong, elliptical, etc. In some embodiments, an outer cross sectional geometry of the adjustable member 104 and an inner cross sectional geometry of the intermediary member 110 may include one or more flat surfaces configured to matingly engage one another. The intermediary member 110 may include at least one groove facing an outer surface of the adjustable member 104 and at least one groove facing an inner surface of the housing 102. A seal 112 (e.g., a radial seal, o-ring, retainer, etc.) may be positioned within the at least one groove facing the outer surface of the adjustable member 104. In some embodiments, a portion of the outer surface of the adjustable member 104 and/or a portion of an internal surface of the housing 102 can be recessed to accommodate the seal 112. That is, the outer surface of the adjustable member 104 and/or a portion of an internal surface of the housing 102 can include complementary grooves facing the grooves formed within the intermediary member 110 for receiving the seal 112 therein. The intermediary member 110, including the seal 112 can help facilitate proper sealing between the housing 102 and the adjustable member 104 so that bodily fluid does not enter the housing 102 when the adjustable implant 100 is implanted. It is to be understood that any combination of seals 112 can be used within the intermediary member 110 without departing from aspects of the disclosure. Further, any of the sealing or retention features described in International Patent Application No. PCT/US2022/031709, filed on Jun. 1, 2022 (and hereby incorporated by reference in its entirety), can also be used without departing from aspects of the disclosure. Additionally, multi-seal configurations can be used to aid in sealing the adjustable member 104 relative to the housing 102, for example, as illustrated and described in U.S. patent application Ser. No. 17/815,694 (filed July 28, 2022), which is hereby incorporated by reference in its entirety.

Turning to FIGS. 2-4, the adjustable implant 100 also includes an actuation assembly 116 positioned within the adjustable member 104 and configured to move the adjustable member 104 within the cavity 131 relative to the housing 102. The actuation assembly 116 includes an actuator 118 configured to drive rotational motion about a rotational axis of the actuator 118. The actuator 118 can take any of a variety of forms such as a motor or an externally driven rotatable permanent magnet. The actuator 118 may be axially fixed within the adjustable member 104 by one or more mechanical hardware components such as one or more bearings. In the embodiment illustrated in FIG. 4, the actuator 118 includes a rotatable magnetic assembly 201 that is located within the adjustable member 104. The magnetic assembly 201 can include a cylindrical, radially-poled permanent magnet 202 contained within a magnet housing 204. The permanent magnet 202 may include rare earth magnet materials, such as Neodymium-Iron-Boron. The permanent magnet 202 can have a protective Phenolic coating and may be held statically within the magnet housing 204 by epoxy or other adhesive. In various implementations, the magnet housing 204 and epoxy form a seal to further protect the permanent magnet 202. The magnet housing 204 may also be welded to create a hermetic seal. Additional details of the magnetic assembly 201 and magnetic housing 204 are included, for example, in U.S. patent application Ser. No. 17/815,694 (filed Jul. 28, 2022), previously incorporated by reference herein, as well as in U.S. Pat. Pub. 20190015138, published Jan. 17, 2019, which is incorporated herein by reference as if set forth in its entirety. Other magnetic assembly members may also be used, such as those disclosed in International Patent App. Pub. No. WO 2022/015898 A1, published Jan. 20, 2022; U.S. Pat. No. 8,734,488, published May 27, 2014; and U.S. Pat. Pub. 20130338714, published Dec. 19, 2013, each of which are incorporated herein by reference as if set forth in its entirety.

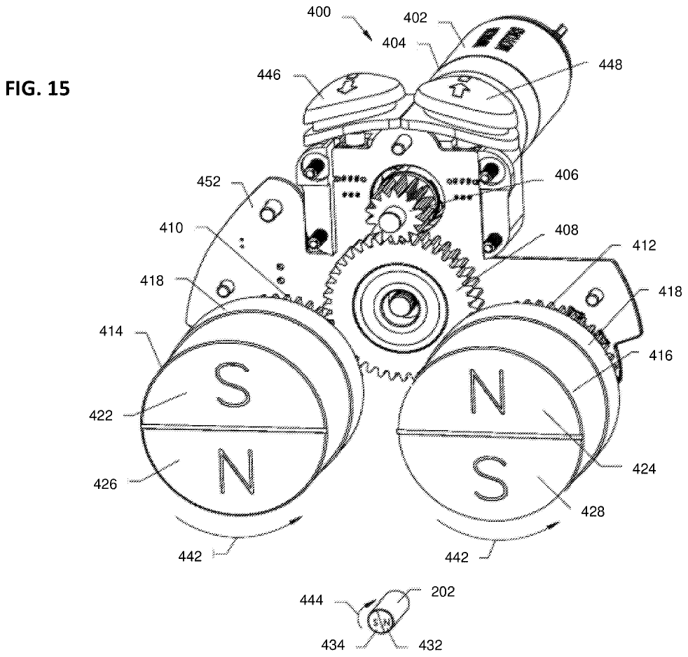

An external adjustment device 400 including an external magnet 414, 416 (see FIGS. 15-17) may be configured to actuate rotation of the actuator 118 in either of a first direction or a second direction about the rotational axis of the actuator 118. Rotation in the first direction may correspond to distraction of the adjustable member 104 and rotation in the second direction may correspond to retraction of the adjustable member 104. For instance, the driver 112 may be configured to rotate about the rotational axis in a first direction corresponding to distal translation of the adjustable member 104 (e.g., distraction), and to rotate in a second direction opposite the first direction corresponding to proximal translation of the adjustable member 104 along the axis (e.g., retraction, as in a compression procedure). Alternatively, the adjustable implant may include a motor configured to rotate in response to an electrical signal (e.g., as provided by an external device). The motor may be electrically coupled to a power source such as an implanted battery or charging capacitor to drive rotation. The power source may be configured for transcutaneous charging using an external power source.

As further shown by FIGS. 2-4, the actuation assembly 116 further includes a gear assembly 120 rotatably coupled to the actuator 118. The gear assembly 120 can include at least one stage of planetary gears. The embodiment illustrated in FIG. 4 includes four stages of gears, including a first stage 224, a second stage 236, a third stage 242, and a fourth stage 244. Although it should be understood that any number of stages may be implemented in various embodiments within the scope of the present disclosure. Each stage of the one or more stages of gears in gear assembly 120 may provide a gear reduction ratio. In various implementations, the four-stage gear assembly 120 enables a higher gear ratio than certain conventional adjustable implants, e.g., those with three-stage gear assemblies. The actuation assembly 116 further includes an output driver 122 rotatably coupled to the gear assembly 120, and a nut 124 disposed at least partially surrounding the output driver 122. The nut 124 may include an outer thread 142 (shown in detail in FIG. 4) configured to communicate with an internal thread 144 of the housing 102. The gear assembly 120 therefore couples to the magnetic assembly 201 at an input end thereof, and couples to the output driver 122 at an opposite, output end thereof, thereby coupling the magnetic assembly 201 to the output driver 122. Rotation of the magnetic assembly 201 therefore rotates the gear assembly 120, which in turn first stage 224, which in turn rotates the second stage 236, which in turn rotates the third stage 242, which in turn rotates the fourth stage 244, and which in turn rotates the output driver 122.

In various implementations, as illustrated in FIG. 4, the adjustable implant 100 further includes a sleeve 300 positioned radially between the actuation assembly 116 and the adjustable member 104. The sleeve 300 can be configured to move independently of the adjustable member 104 to enable measurement of a load on the adjustable implant 100. In certain implementations, the sleeve 300 surrounds the actuation assembly 116, e.g., around the axis of displacement of the adjustable implant 100. In particular cases, the sleeve 300 is circumferentially contiguous and wraps around the actuation assembly 116. In particular implementations, the sleeve 300 includes an anti-rotation mechanism 301 that enables the sleeve 300 to move axially independently of the adjustable member 104 without substantial rotation about the distraction axis.

In some implementations, the sleeve 300 includes a load cell 302 that is contained in the adjustable member 104. In certain cases, the load cell 302 is coupled with an end 304 of the actuation assembly 116. The load cell 302 can permit measurement of a load on the adjustable implant 100 from inside the adjustable implant 100. That is, the load cell 302 can enable measurement of the load on the adjustable implant 100 from a measurement location that is internal to the adjustable implant 100. In particular cases, the load cell 302 enables direct measurement of the internal load on the adjustable implant 100. Further, in contrast to conventional adjustable implants, the load cell 302 permits measurement of the load on the adjustable implant 100 without a direct measurement of load on a bone anchor (e.g., connected at apertures 106) coupling the adjustable implant 100 with a bone of a patient. In various implementations, the load cell 302 permits measurement of both a compressive load and a tensile load on the adjustable implant 100. That is, the load cell 302 can provide both compressive load data and tensile load data for use in distraction and/or compression applications.

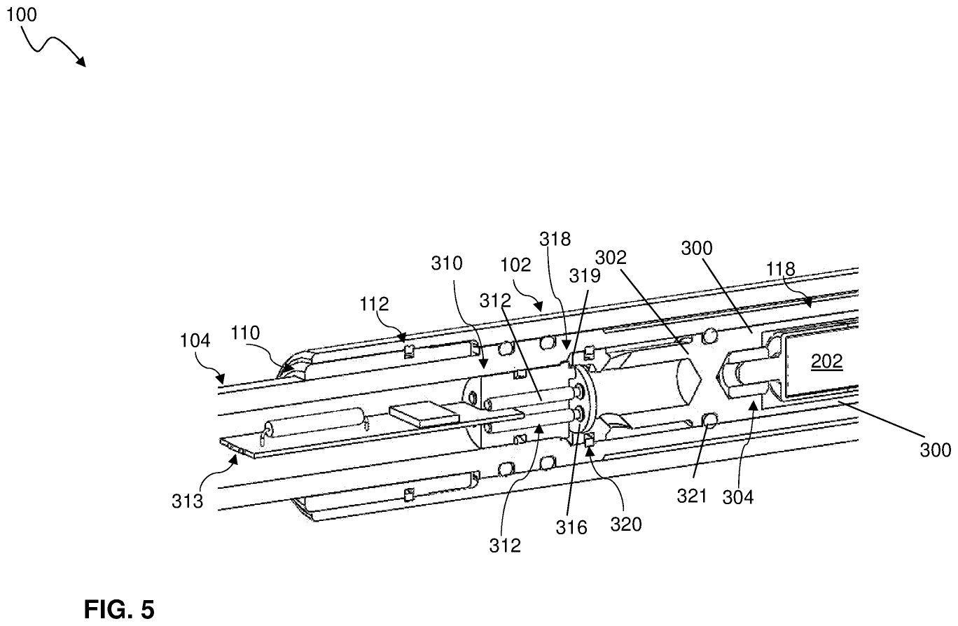

As shown in FIG. 4, as well as the additional sectional view of the adjustable implant 100 in FIG. 5, a strain gage (or, gauge) 310 can be connected with the load cell 302 by a set of interconnections 312. In various implementations, the strain gauge 310 includes a circuit 313 configured to calculate a load value from detected strain. In some cases, an alignment pin 316 couples the strain gauge 310 with the load cell 302. In various implementations, the adjustable member 104 includes a shoulder 318 that is engaged with the alignment pin 316. In particular cases, the shoulder 318 engages an end 319 of the sleeve 300, which surrounds a portion of the alignment pin 316. Further, a retainer 320 can couple the load cell 302 with the adjustable member 104. In particular cases, one or more seals 321 (e.g., o-rings) are located within slots in the load cell 302 to aid in sealing the mechanism. Because the load cell 302 is coupled with both the sleeve 300 and the end 304 of the actuation assembly 116, the load cell 302 is positioned to measure the bi-directional load on the adjustable implant 100 based on relative load on the floating sleeve 300.

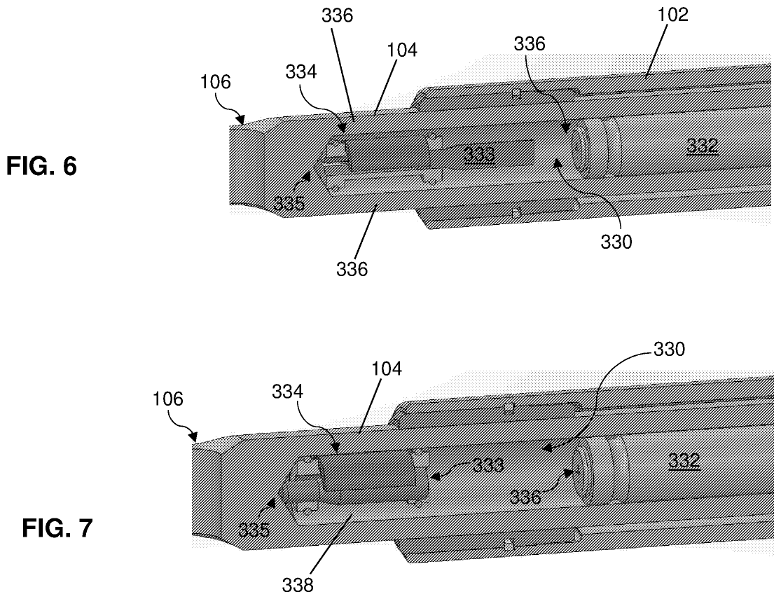

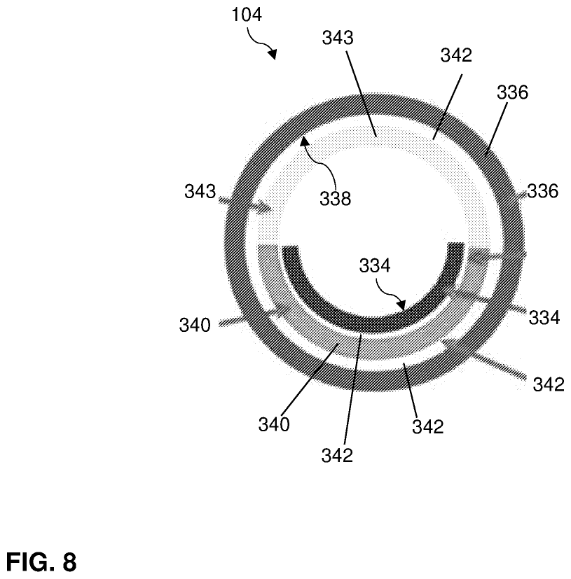

In further implementations, as illustrated in FIGS. 6 and 7, the adjustable implant 100 further includes a battery slot 330 in the adjustable member 104 configured to receive a battery 332. In various implementations, the battery 332 is connected with a transducer 334. In certain example implementations, the transducer 334 includes an ultrasound transducer, e.g., a piezoelectric transducer. However, other transducer types can be used, for example, a magnetostrictive transducer. In various implementations, the transducer 334 is coupled (e.g., via wired connection) with the strain gauge 310 to enable receiving strain data and transmitting an indicator of strain data externally from the adjustable implant 100. In various implementations, an expander (or connector) 333 is configured to contact a positive terminal 336 of the battery 332. According to certain implementations, a conductive bonding material (e.g., epoxy) 342 (FIG. 8) is present along the outer radii of the transducer 334, and functions as a negative (or, ground) path. In certain cases, the battery 332 is wired to the circuit 313 (e.g., PCB board).

As described herein, the transducer 334 can enable communication with an external device (e.g., receiver and/or controller such as the external adjustment device 400, FIG. 15) to provide load data about the adjustable implant 100. In a particular implementation, the transducer 334 is positioned such that the housing 102 does not obstruct signal transmission to or from the transducer 334 while the adjustable implant 100 is in a fully retracted state, e.g., as illustrated in FIGS. 6 and 7. In certain example implementations, the transducer 334 (e.g., piezoelectric transducer) is partially arcuate to enable directional control of ultrasonic signals. For example, the partially arcuate (e.g., piezoelectric) transducer 334 has a tuned signal strength in at least one direction. In other terms, the transducer 334 can have a greater signal strength in at least one direction relative to another direction, e.g., when considered radially, or approximately perpendicular to the direction of distraction/compression.

In certain examples, e.g., as illustrated in FIGS. 6 and 7, the transducer 334 abuts an outer wall 336 of the adjustable member 104. For example, the transducer 334 can abut the outer wall 336 of the adjustable member with only a bonding material therebetween (e.g., an epoxy). In certain of these examples, the transducer 334 is bonded directly to an inner surface 338 of the outer wall 336 without an air gap therebetween. This configuration can enhance signal strength in communication with the transducer 334, as air gap(s) can notably diminish the strength of ultrasonic signals traveling therethrough. In another implementation, as illustrated in the cross-sectional view of the adjustable implant 100 in FIG. 8, the (e.g., piezoelectric) transducer 334 is bonded to the inner surface 338 of the outer wall 336 with a titanium sleeve 340 therebetween, e.g., with one or more layers of bonding material 342 (e.g., epoxy such as silver epoxy). In various implementations, the bonding material 342 is conductive, i.e., enabling ultrasound transmission therethrough. In certain cases, an alignment sleeve 343 is coupled with the titanium sleeve 340 and spans partially annularly around the transducer 334. The alignment sleeve 343 can aid in aligning the titanium sleeve 340 adjacent to the transducer 334, e.g., to enable directional communication to/from the transducer 334.

As noted herein, the transducer 334 is an internal component to the adjustable implant 100. At least in part because of the location of the transducer 334 (within the battery slot 330 defined by the outer wall 336 of the adjustable member 104) while the implant is in the fully retracted position (e.g., FIG. 2), various implementations include a process of positioning the transducer 334 in the adjustable member. In certain cases, a bonding material 342 is applied to the transducer 334 (or any intervening sleeve(s), e.g., titanium sleeve 340), and the transducer 334 is then loaded from an opening in the adjustable member 104 to a base 335 of the battery slot 330 in the adjustable member 104. The bonding material 342 with the coupled transducer 334 can then be directly applied to the inner surface 338 of the outer wall 336 to bond the transducer 334 to the adjustable member 104. In some cases, a telescoping tool or elongated tool is used to load the transducer 334 into the battery slot 330. In various implementations, the bonding material 342 can include a self-curing material or an adhesive. In particular examples, the bonding material 342 includes an epoxy that is applicable with an approximately consistent thickness and no more than nominal air bubbles. In certain cases, the bonding material 342 is applied selectively to avoid contact with the positive inner surface of the transducer 334.

The adjustable implant 100 can include various additional beneficial features for enhancing use and/or the patient experience. For example, in additional implementations, as illustrated in FIGS. 3 and 4, the intermediary member 110 can be configured to function as an anti-rotation lug to limit rotation of the adjustable member 104 relative to the housing 102. For example, as noted herein, the intermediary member 110 can at least partially surround the adjustable member 104 and include one or more flat or non-rounded surfaces that function to limit rotation relative to the housing 102.

Further, in certain cases, threads 142 in housing 102 (FIG. 4) can have distinct thread pitches to control a distraction rate of the actuation assembly 116. For example, a first subset of the threads 142 has a first thread pitch, and a second subset of the threads 142 has a second, distinct thread pitch, enabling distinct distraction rates of the actuation assembly 116 when engaged with distinct subsets of threads.

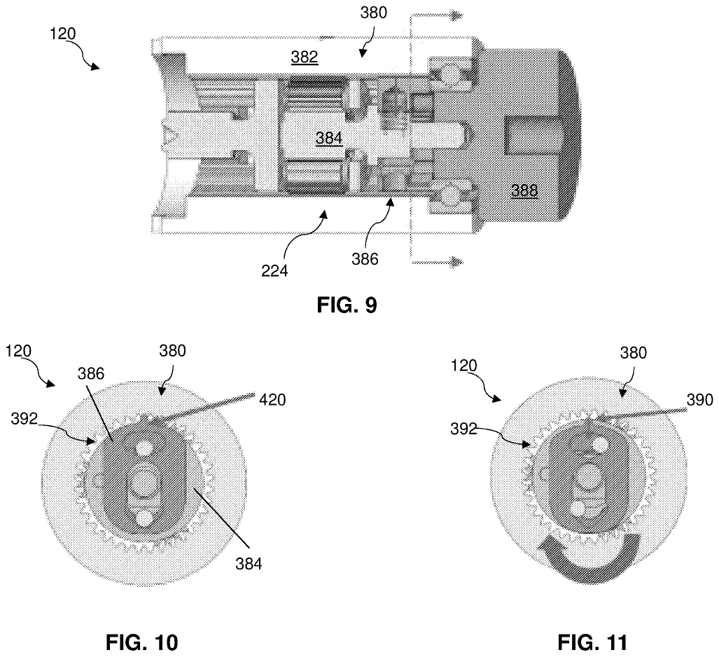

As noted herein with respect to FIGS. 1-4, the gear assembly 120 can be controlled by the actuator 118. In some cases, e.g., where the actuator 118 includes a magnetic actuator, as illustrated in FIGS. 9-11, the adjustable implant 100 includes a locking mechanism 380 coupled with the gear assembly 120 for controlling distraction loss of the actuation assembly 116. In these cases, the magnetic actuator may be susceptible to distraction loss, and the locking mechanism 380 can mitigate that distraction loss. FIG. 9 shows a side cross-section of a portion of the gear assembly 120 including the locking mechanism 380, including a fixed ring gear 382, a keyed drive gear 384, and a keeper 386 that is configured to engage/disengage (or lock/unlock) to function as a distraction loss mechanism. Engaging and disengaging the ring gear 382 can be controlled by actuation of a magnet 388, e.g., rotation of the magnet 388 to engage/disengage the ring gear 382. A tab (or, protrusion) 390 on the keeper 386 can be configured to engage/disengage teeth 392 in the ring gear 382 to engage/disengage the locking mechanism 380. FIG. 10 illustrates the locking mechanism 380 in a locked (engaged) position, and FIG. 11 illustrates the locking mechanism 380 in an unlocked (disengaged) position. Additional details of this locking mechanism 380 are described and illustrated in U.S. patent application Ser. No. 17/815,694 (previously incorporated by reference herein). In other implementations, the actuator 118 includes a motor with an integrated distraction loss mechanism.

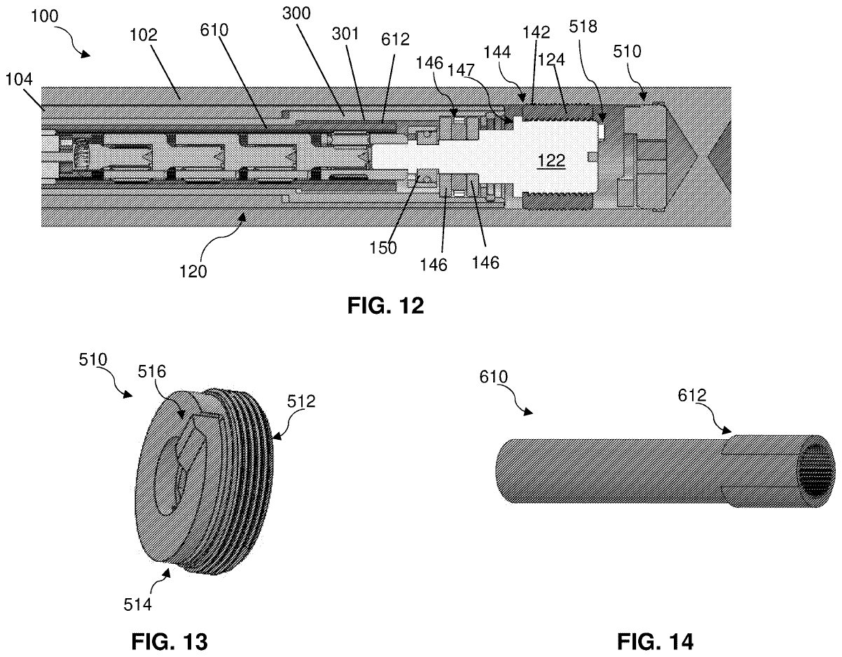

In various additional implementations, the adjustable implant 100 is configured to mitigate jamming (or, sticking) during use. For example, as illustrated in FIGS. 12-14, the adjustable implant 100 can include an anti-jam member 510 in the cavity 131 for preventing the adjustable member 104 from getting jammed or otherwise undesirably caught in the housing 102. The anti-jam member 510 can include a set of threads 512 for engaging the internal threads 144 in the housing, along with a spring mechanism 514 with a tab 516 for selectively engaging a protrusion 518 on the output driver 122. Further features of the anti-jam member 510 are described and illustrated, e.g., in U.S. patent application Ser. No. 17/815,694 (previously incorporated by reference herein).

In further implementations, as noted herein, the gear assembly 120 can include one or more features to enhance beneficial operation of the adjustable implant 100. For example, the gear assembly 120, which can include a set of gear stages (e.g., four stages), can include one or more sun gears, planetary gears and/or ring gears. In a particular example shown in FIGS. 12 and 14, the gear assembly 120 includes a ring gear 610 that has an anti-rotation feature 612. The anti-rotation feature 612 can include one or more flat or non-rounded sections that prevent the ring gear 610 from rotating relative to the sleeve 300. Additionally, as illustrated in FIG. 12, the implant can further one or more thrust bearings 146 and associated retainer clips 150. In some embodiments, a thrust bearing 146 is positioned about at least a portion of the output driver 122 within the adjustable member 104. The thrust bearings 146 serve to protect the magnetic assembly 201 (FIG. 4) and the gear assembly 120 of the drive system from any significant compressive or tensile stresses. When there is a compressive force on the adjustable implant 100, for example, when distracting a bone, and thus resisting the tensile strength of the soft tissues, the thrust bearing(s) 146 abut against the retainer clip(s) 150 and/or a ledge 147 on the output driver 122. For example, in certain compressive applications it is the goal to hold two fractured sections of a bone together. Because the bone may have fractured in a non-uniform or shattered pattern, it may be difficult to determine the desired length of the adjustable implant 100 until after it is implanted and fully attached. In these situations, it may be preferred to place a slightly extended adjustable implant 100, secure the adjustable implant 100, and then magnetically retract the adjustable implant 100 after it has been secured within the bone fragments. In this manner, the adjustable implant 100 may apply the desired compression between the two fragments.

In such compressive applications, the adjustable implant 100 is under tensile forces and the thrust bearing(s) 146 would abut against the retainer clip(s) 150 or a ledge 147. In both situations, the thrust bearings 146 and the ledge 147 take the large stresses, rather than the magnetic assembly 201 or gear assembly 120 of the drive system. Additional details of example gear stages and components is included, e.g., in U.S. patent application Ser. No. 17/815,694 (previously incorporated by reference herein).

FIGS. 15-17 illustrate an external adjustment device 400 configured for applying a moving magnetic field to allow for non-invasive adjustment of the adjustable implant 100 by turning a permanent magnet 202 within the adjustable implant 100, as described. FIG. 15 illustrates the internal components of the external adjustment device 400, and for clear reference, shows the permanent magnet 202 of the adjustable implant 100 without the rest of the assembly. The internal working components of the external adjustment device 400 may, in certain embodiments, be similar to that described in U.S. Patent Application Publication No. 2012/0004494, which is incorporated by reference herein. A motor 402 with a gear box 404 outputs to a motor gear 406. Motor gear 406 engages and turns central (idler) gear 408, which has the appropriate number of teeth to turn first and second magnet gears 410, 412 at identical rotational speeds. First and second magnets 414, 416 turn in unison with first and second magnet gears 410, 412, respectively. Each magnet 414, 416 is held within a respective magnet cup 418 (shown partially). An exemplary rotational speed is 60 RPM or less. This speed range may be desired in order to limit the amount of current density induced in the body tissue and fluids, to meet international guidelines or standards. As seen in FIG. 15, the south pole 422 of the first magnet 414 is oriented the same as the north pole 424 of the second magnet 416, and likewise, the first magnet 414 has its north pole 426 oriented the same as the south pole 428 of the second magnet 416. As these two magnets 414, 416 turn synchronously together, they apply a complementary and additive moving magnetic field to the radially-poled, permanent magnet 202, having a north pole 432 and a south pole 434. Magnets having multiple north poles (for example, two) and multiple south poles (for example, two) are also contemplated in each of the devices. As the two magnets 414, 416 turn in a first rotational direction 442 (e.g., counter-clockwise), the magnetic coupling causes the permanent magnet 202 to turn in a second, opposite rotational direction 444 (e.g., clockwise). The rotational direction of the motor 402 and corresponding rotational direction of the magnets 414, 416 is controlled by buttons 446, 448. One or more circuit boards 452 contain control circuitry for both sensing rotation of the magnets 414, 416 and controlling the rotation of the magnets 414, 416.

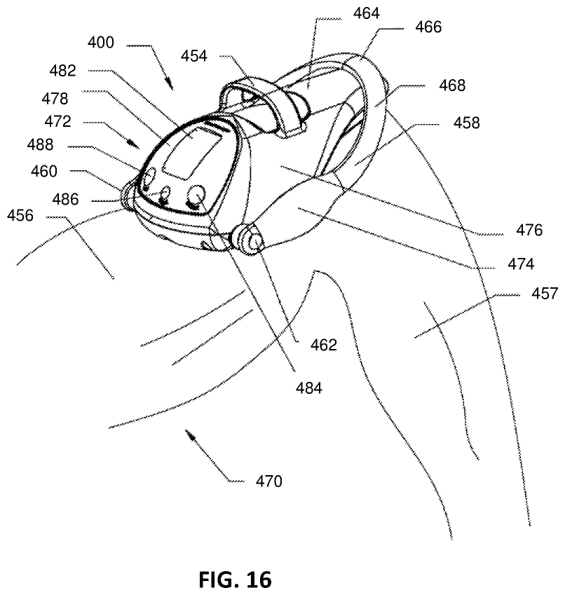

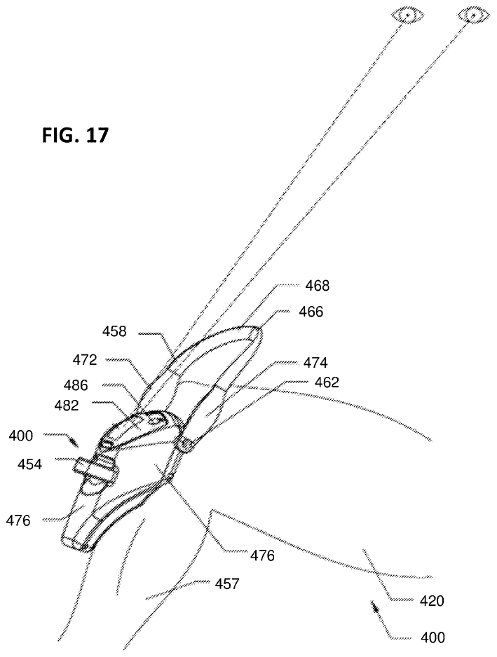

FIGS. 16 and 17 show the external adjustment device 400 for use with an adjustable implant 100 placed in the femur (FIG. 16) or the tibia (FIG. 17) or an adjustable implant placed along a spinal curvature. The external adjustment device 400 has a first handle 454 for carrying or for steadying the external adjustment device 400, for example, steadying it against an upper leg 456 (as in FIG. 16) or lower leg 457 as in (FIG. 17). An adjustable handle 458 is rotationally attached to the external adjustment device 400 at pivot points 460, 462. Pivot points 460, 462 have easily lockable/unlockable mechanisms, such as a spring-loaded brake, ratchet, or tightening screw, so that a desired angulation of the adjustable handle 458 in relation to the housing 464 can be adjusted and locked in orientation. The adjustable handle 458 is shown in two different positions in FIGS. 16 and 17. In FIG. 16, the adjustable handle 458 is set so that the apex 466 of the loop 468 rests against the housing 464. In this position, the patient 470 is able to hold onto one or both of the grips 472, 474 while the adjustment procedure (for example transporting bone between 0.10 mm to 1.50 mm) is taking place. It is contemplated that the procedure could also be a lengthening procedure for a bone lengthening device or a lengthening procedure for a lengthening plate which is attached external to the bone. Turning to FIG. 17, when the adjustable implant 100 is implanted in a tibia, the adjustable handle 458 may be changed to a position in which the patient 470 can grip onto the apex 466 so that the magnet area 476 of the external adjustment device 400 is held over the portion the adjustable implant 100 containing the permanent magnet 202. In both cases, the patient 470 is able to clearly view the control panel 478 including a display 482. In a different configuration from the two directional buttons 414, 416 in FIG. 15, the control panel 478 includes a start button 484, a stop button 486 and a mode button 488. Control circuitry contained on circuit boards 452 may be used by the surgeon to store important information related to the specific aspects of each particular patient.

For example, in some patients an implant may be placed antegrade into the tibia. In other patients the implant may be placed either antegrade or retrograde about the femur. In still other patients, the implant may be placed about an area of curvature in the spine of the patient. In each of these cases, it may be desired to move the bone either from distal to proximal or from proximal to distal. By having the ability to store information of this sort that is specific to each particular patient within the external adjustment device 400, the external adjustment device 400 can be configured to direct the magnets 414, 416 to turn in the correct direction automatically, while the patient need only place the external adjustment device 400 at the desired position, and push the start button 484. The information of the maximum allowable bone transport length per day and maximum allowable bone transport length per session can also be input and stored by the surgeon for safety purposes. These may also be added via an SD card or USB device, or by wireless input. An additional feature is a camera at the portion of the external adjustment device 400 that is placed over the skin. For example, the camera may be located between first magnet 414 and second magnet 416. The skin directly over the implanted permanent magnet 202 may be marked with indelible ink. A live image from the camera is then displayed on the display 482 of the control panel 478, allowing the user to place the first and second magnets 414, 416 directly over the area marked on the skin. Crosshairs can be overlaid on the display 482 over the live image, allowing the user to align the mark on the skin between the crosshairs, and thus optimally place the external adjustment device 400.

Other external adjustment devices can be used to cause actuation of the distraction devices described herein. Such external adjustment devices include, for example, those described in U.S. Pat. No. 8,382,756, published on Feb. 26, 2013; U.S. Pat. No. 9,248,043, published on Feb. 2, 2016; U.S. Pat. No. 9,078,711, published on Jul. 14, 2015; U.S. Pat. No. 9,044,281, published on Jun. 2, 2015; U.S. Pat. No. 11,246,694, published on Feb. 15, 2022; U.S. Pat. App. Pub. No. 2016/0113683 A1, published on Apr. 28, 2016; U.S. Pat. No. 10,835,290, published on Nov. 17, 2020; and International Patent App. No. PCT/US2020/017338, published as WO 2020/163800 A1 on Aug. 13, 2020, all of which are incorporated herein by reference as if set forth in their entirety.

In addition to approaches for actuating the actuator 118 to adjust the adjustable implant 100, various approaches can include measuring the load on the adjustable implant 100, e.g., via the transducer 334 and the load cell 302. In these cases, the external adjustment device 400 (or another computing device such as a smartphone, tablet, etc.) can be configured to measure the load on the adjustable implant 100. In such cases, the external adjustment device 400 (or other computing device) is configured to receive an indicator of a load on the adjustable implant 100 via the transducer 334. In certain cases, the external adjustment device 400 is configured to compare that indicator of the load (e.g., load data such as one or more load values or range(s) of loads) with a load threshold. In some cases, the load threshold is patient-specific, device specific (e.g., based on the size of implant 100), and/or treatment-plan specific (e.g., based on an aggregate adjustment schedule over a period). In some aspects, where the indicator of the load deviates from the threshold, an approach can include actuating the actuator 118 to adjust the adjustable implant 100. In particular examples, iterative adjustments can be made to the adjustable implant 100 via the actuator 100 based on successive indicators of load, e.g., via transducer 334 and load cell 302, until the indicator of load satisfies the threshold(s).

In any case, in contrast to certain conventional implants and approaches, various disclosed embodiments allow for efficient and effective intramedullary bone adjustment. The disclosed implants can have a compact footprint while enabling high gear ratio adjustment for flexibility in adjustment. Further, the disclosed implants can provide precise load data (e.g., wirelessly) with a direct load measurement from the implant. This load data can be used to more precisely assess aspects of the bone adjustment as well as stresses on the implant. Further, the disclosed implants can enable effective communication of load data via a transducer configuration that enhances signal transmission and quality. These and other features (e.g., anti-rotation features, anti-jam features) provide an adjustable implant that enhances the patient experience and improves procedural outcomes for the provider.

Additionally, the load data provided by the disclosed implants can help medical professionals (e.g., surgeons) to assess bone regeneration quality, and establish and/or modify treatment protocols. For example, if the distraction rate for the implant is too slow (as indicated by reduced load on the implant corresponding with increased calcification), the surgeon can modify the distraction rate accordingly. Further, once the load on the implant reaches a threshold (e.g., lower threshold), that load value may indicate desired healing (or calcification). The surgeon can then order a verification of the healing (e.g., via imaging such as an x-ray), and subsequently remove the implant if the healing is confirmed.

While implementations above are primarily in the context of externally magnetically driven systems, other drive systems can be used. For example, in addition to or instead of the magnet-based driving, one or more of the drive elements can take the form of an implanted electric motor. The implanted electric motor can be powered by an external power source (e.g., via a radiofrequency link, via an ultrasonic energy transfer technique, via an inductive connection, via another technique, or via combinations thereof) or an implanted power source (e.g., a battery, which may be charged by the external power source). The implanted power source may be within the implant (e.g., within a housing thereof) or separate from the implant and coupled to the implant via a cable.

Commonly labeled components in the FIGURES are considered to be substantially equivalent components for the purposes of illustration, and redundant discussion of those components is omitted for clarity. Further, as used herein, the term “approximately”can refer to a defined value, plus or minus several percent.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B; ” “one or more of A and B; ” and “A and/or B” are each intended to mean “A alone, B alone, or A and B together. ” A similar interpretation is also intended for lists including three or more items. For example, the phrases “at least one of A, B, and C; ” “one or more of A, B, and C; ” and “A, B, and/or C” are each intended to mean “A alone, B alone, C alone, A and B together, A and C together, B and C together, or A and B and C together. ” Use of the term “based on,” above and in the claims is intended to mean, “based at least in part on,” such that an unrecited feature or element is also permissible.

The terminology used herein is for the purpose of describing particular embodiments only and is not intended to be limiting of the disclosure. As used herein, the terms “first,” “second,” and the like, do not denote any order, quantity, or importance, but rather are used to distinguish one element from another, and the terms “a” and “an” herein do not denote a limitation of quantity, but rather denote the presence of at least one of the referenced item. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups. As used herein, “substantially” refers to largely, for the most part, entirely specified or any slight deviation which provides the same technical benefits of the disclosure.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and subcombinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

We claim:

1. An adjustable implant comprising:
a housing having an internal thread and defining a cavity within the housing;
an adjustable member at least partially positioned within the housing and moveable relative to the housing within the cavity, the adjustable member including a transducer adjacent an outer wall thereof;
an actuation assembly positioned within the adjustable member and configured to move the adjustable member relative to the housing, the actuation assembly including:
an actuator configured to be activated by an external adjustment device;
a gear assembly coupled to the actuator; and
an output driver coupled to the gear assembly; and
a sleeve located radially between the actuation assembly and the adjustable member, the sleeve including a load cell coupled with the transducer.

2. The adjustable implant of claim 1, wherein the transducer is configured to transmit signals about a load on the adjustable implant detected by the load cell.

3. The adjustable implant of claim 1, wherein the sleeve is configured to move independently of the adjustable member to enable measurement of a load on the adjustable implant.

4. The adjustable implant of claim 1, further comprising a battery slot in the adjustable member and a battery in the battery slot.

5. The adjustable implant of claim 4, wherein the transducer is connected with the battery.

6. The adjustable implant of claim 1, wherein the transducer is positioned such that the housing does not obstruct signal transmission to or from the transducer while the adjustable implant is in a fully retracted state.

7. The adjustable implant of claim 1, wherein the transducer includes an ultrasound transducer.

8. The adjustable implant of claim 7, wherein the ultrasound transducer includes a piezoelectric transducer.

9. The adjustable implant of claim 8, wherein the piezoelectric transducer is partially arcuate to enable directional control of ultrasonic signals.

10. The adjustable implant of claim 9, wherein the piezoelectric transducer has a tuned signal strength in at least one direction.

11. The adjustable implant of claim 8, wherein the piezoelectric transducer abuts an outer wall of the adjustable member.

12. The adjustable implant of claim 11, wherein the piezoelectric transducer is bonded to an inner surface of the outer wall of the adjustable member without an air gap therebetween.

13. A method comprising: positioning the piezoelectric transducer of claim 8 in the adjustable member.

14. The method of claim 13, further comprising bonding the piezoelectric transducer to an inner surface of an outer wall of the adjustable member.

15. A method comprising: measuring the load on the adjustable implant of claim 1.

16. The method of claim 50, further comprising actuating the actuator to adjust the adjustable implant based on the measured load.
